(19) United States
(12) Patent Application Publication
(10) Pub. No.: US 2026/0090816 A1
(43) Pub. Date: Apr. 2, 2026

(54) Steerable Shaft Assistance Control

(71) Applicant: COOK MEDICAL TECHNOLOGIES LLC, Bloomington, IN (US)

(72) Inventors: William B. Key, Pittsboro, NC (US); Alexander B. Thomason, Advance, NC (US); Jonathan P. Delemar, Winston-Salem, NC (US); Nadia M. Hepburn, Winston-Salem, NC (US); Christopher A. Carruthers, Winston-Salem, NC (US); Shaun D. Gittard, Winston-Salem, NC (US)

(21) Appl. No.: 19/341,409

(22) Filed: Sep. 26, 2025

Related U.S. Application Data
(60) Provisional application No. 63/700,001, filed on Sep. 27, 2024.

(51) Int. Cl. A61B 17/29; A61B 1/00; A61B 1/005; A61B 17/00

(52) U.S. Cl. CPC A61B 17/2909; A61B 1/00131; A61B 1/005; A61B 2017/00477

(57) ABSTRACT

A control assembly for a steerable medical device shaft, the control assembly may include a steerable-shaft handle; and an elongate docking translating base, including a first-end opening dimensioned to receive and operably engage a portion of a separate medical device handle and a second-end ball. The steerable-shaft handle surrounds a longitudinal passage extending distally from a first-end terminus that includes a socket dimensioned to engagingly receive the second-end ball. The longitudinal passage includes a path in mechanical communication with the first-end opening of the base. The base includes at least two steering wires that extend through the steerable-shaft handle in mechanical communication with a steerable length of a shaft length disposed distal of the steerable-shaft handle.

### CROSS-REFERENCE TO RELATED APPLICATIONS

The present application claims the benefit of U.S. Provisional Application No. 63/700,001, filed on Sep. 27, 2024, the teachings of which are incorporated herein by reference.

### BACKGROUND

The statements in this section merely provide background information related to the present disclosure and may not constitute prior art.

The traditional endoscope is a medical device used in a variety of procedures. For example, a physician may insert the endoscope, for example, into a patient's mouth or into another body opening and then manipulate the distal end of the device through the patient's gastrointestinal (GI) tract to perform a particular endoscopic procedure. The physician may then use a variety of instruments during the procedure that are passed through an accessory channel that is located within the outer shaft of the endoscope. As the endoscopy field advances, new endoscopes are being created for specific procedures.

Endoscopes are typically steerable from a proximal-end handle. For example, certain steering features and mechanisms for controlling the distal end of the endoscope are discussed in U.S. Patent Application Pub. No. 2015/0366435, which is hereby incorporated by reference in its entirety. While steerable endoscopes are used with success to treat a variety of issues, existing endoscopes are often challenging to steer when inside the body, particularly when the outer shaft of the endoscope is steered separately from components extending through the accessory channel (e.g., meaning two separate steering systems are needed).

During an endoscopic procedure, the physician needs to execute various controls and movements, so it is desired to allow another person (e.g., physician's assistant) to manage the steering of a separate steerable component. The present disclosure presents an improved steering system for use with a variety of endoscopes where the steering system may allow a physician or physician's assistant to control the steering of a separate steerable component extending through the accessory channel and manipulate another medical device (e.g., biopsy accessory) provided by the separate steerable component (e.g., at least partially extending through the separate steerable component) simultaneously, via one or two hands.

### SUMMARY

One general aspect of the present disclosure includes a control assembly for a steerable medical device shaft, the control assembly including: a steerable-shaft handle; and an elongate docking translating base, including a first-end lengthwise opening dimensioned to receive and operably engage a portion of a separate medical device handle and a second-end ball with an outer diameter larger than an adjacent lengthwise portion of the base, where the steerable-shaft handle coaxially surrounds a longitudinal passage extending distally from a first-end terminus that includes a socket dimensioned to engagingly receive the second-end ball, the longitudinal passage including a path in mechanical communication with the first-end lengthwise opening of the base, and where the base includes at least two steering wires that extend through the steerable-shaft handle in mechanical communication with a steerable length of a shaft length disposed distal of the steerable-shaft handle.

Another general aspect of the present disclosure includes a control assembly for a steerable medical device shaft, the control assembly including: a steerable-shaft handle; and an elongate docking translating base, including a first-end lengthwise opening dimensioned to receive and frictionally engage a portion of a separate medical device handle and a second-end ball with an outer diameter larger than an adjacent lengthwise portion of the base, where the elongate docking translating base includes a first portion and a second portion, the first portion being selectively moveable relative to the second portion and selectively lockable to the second portion, where the steerable-shaft handle surrounds a longitudinal passage extending distally from a first-end terminus that includes a socket dimensioned to engagingly receive the second-end ball, and where the base includes at least two steering wires that extend through the steerable-shaft handle in mechanical communication with a steerable length of a shaft length disposed distal of the steerable-shaft handle.

Another general aspect of the present disclosure includes a scope system, including: a steerable outer sheath including a lumen extending therethrough and an articulating distal lengthwise portion; and an accessory channel shaft including an accessory lumen extending therethrough and a distal lengthwise section, the accessory channel shaft movably disposed at least partially within the lumen of the steerable outer sheath, where the accessory channel shaft is moveable between a forward-viewing configuration and a side-viewing configuration relative to the steerable outer sheath, where, in the forward-viewing configuration, the distal lengthwise section of the accessory channel shaft is parallel to and/or disposed at least partially within the distal lengthwise portion of the steerable outer sheath, where, in the side-viewing configuration, the distal lengthwise section of the accessory channel shaft is disposed at a non-parallel angle relative to the distal lengthwise portion of the steerable outer sheath, where a steerable medical device shaft is movably disposed in the accessory channel shaft, and where the steerable medical device shaft is controlled by a control assembly including a steerable-shaft handle, the steerable-shaft handle is connected to a docking translating base, and the base includes a first-end opening dimensioned to receive and operably engage a portion of a separate medical device handle.

A control assembly for a steerable medical device shaft according to the present disclosure may include any combination of the features described above and/or the original as-filed claims.

Other systems, methods, features and advantages of the invention will be, or will become, apparent to one with skill in the art upon examination of the following figures and detailed description. It is intended that all such additional systems, methods, features and advantages be within the scope of the invention.

### BRIEF DESCRIPTION OF THE DRAWINGS

In order that the present disclosure may be well understood, there will now be described various forms thereof, given by way of example, reference being made to the accompanying drawings. The components in the figures are not necessarily to scale. Moreover, in the figures, like-referenced numerals designate corresponding parts throughout the different views.

FIG. 1 is an illustration showing a medical device having a steerable portion in accordance with certain aspects of the present disclosure.

FIG. 2 is an illustration showing the steerable medical device, also having an additional steerable shaft for delivering an endoscopic instrument in accordance with certain aspects of the present disclosure.

FIG. 3A is an illustration showing a view of a steerable shaft, and particularly a deflectable tip in accordance with certain aspects of the present disclosure.

FIG. 3B is an illustration showing a view of a steerable shaft, and particularly a deflectable tip in accordance with certain aspects of the present disclosure.

FIG. 3C is an illustration showing a views of a steerable shaft, and particularly a deflectable tip in accordance with certain aspects of the present disclosure.

FIG. 4 is an illustration showing an embodiment of a control assembly for a steerable shaft.

FIG. 5 is an illustration showing a disassembled control assembly of FIG. 4.

FIG. 6 is an illustration showing a disassembled docking translating base of the control assembly of FIG. 4, as well as steering wires.

FIG. 7 is an illustration showing an inner configuration of a portion of the assembled docking translating base of FIG. 6.

FIG. 8 is an illustration showing an inner configuration of a portion of the assembled docking translating base of FIG. 6.

FIG. 9 is an illustration showing a portion of a docking translating base of a control assembly for a steerable shaft and a separate medical device handle in accordance with certain aspects of the present disclosure.

FIG. 10 is an illustration showing a retractable grip of a control assembly for a steerable shaft in accordance with certain aspects of the present disclosure.

FIG. 11 is an illustration showing a retractable grip of a control assembly for a steerable shaft in accordance with certain aspects of the present disclosure.

FIG. 12 is an illustration showing the connection between a first portion and a second portion of the docking translating base of FIG. 6, as well as steering wires and a tensioning control mechanism of the steering wires in accordance with certain aspects of the present disclosure.

FIG. 13 is an illustration showing the connection between a first portion and a second portion of the docking translating base of FIG. 6, as well as steering wires and a tensioning control mechanism of the steering wires in accordance with certain aspects of the present disclosure.

FIG. 14 is an illustration showing the connection between a first portion and a second portion of the docking translating base of FIG. 6, as well as steering wires and a tensioning control mechanism of the steering wires in accordance with certain aspects of the present disclosure.

FIG. 15 is an illustration showing the connection between a first portion and a second portion of the docking translating base of FIG. 6, as well as steering wires and a tensioning control mechanism of the steering wires in accordance with certain aspects of the present disclosure.

FIG. 16 is an illustration showing another tensioning control mechanism of the steering wires in accordance with certain aspects of the present disclosure.

FIG. 17 is an illustration showing another view of the tensioning control mechanism of FIG. 16 in accordance with certain aspects of the present disclosure.

FIG. 18 is an illustration showing another view of the tensioning control mechanism of FIG. 16 in accordance with certain aspects of the present disclosure.

FIG. 19 is an illustration showing another tensioning control mechanism of the steering wires in accordance with certain aspects of the present disclosure.

FIG. 20 is an illustration showing another tensioning control mechanism of the steering wires in accordance with certain aspects of the present disclosure.

FIG. 21 is an illustration showing another tensioning control mechanism of the steering wires in accordance with certain aspects of the present disclosure.

FIG. 22 is an illustration showing another tensioning control mechanism of the steering wires in accordance with certain aspects of the present disclosure.

FIG. 23 is an illustration showing another tensioning control mechanism of the steering wires in accordance with certain aspects of the present disclosure.

### DETAILED DESCRIPTION

The following description is merely exemplary in nature and is not intended to limit the present disclosure, application, or uses. It should be understood that throughout the drawings, corresponding reference numerals indicate like or corresponding parts and features.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer to an axial direction that aligns with the proximal-distal axis of the device (or component), for example, when the device is not bent. The terms “radially” and “radial” will be used to refer to elements, surfaces, or assemblies relative to one another that may extend perpendicularly from a longitudinal axis. The terms “circumference,” “circumferentially,” and “circumferential” will be used to elements, surfaces, or assemblies relative to one another encircling or substantially encircling a longitudinal axis at a radius.

The uses of the terms “a” and “an” and “the” and similar references in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by the Applicant to the contrary, to mean a quantity of more than one. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, the terms “comprise(s),” “include(s),” “having,” “has,” “can,” “contain(s),” and variants thereof, are intended to be open-ended transitional phrases, terms, or words that do not preclude the possibility of additional acts or structures. The present description also contemplates other examples “comprising,” “consisting of,” and “consisting essentially of” the elements presented herein, whether explicitly set forth or not.

In describing elements of the present disclosure, the terms 1st, 2nd, first, second, A, B, (a), (b), and the like, may be used herein. These terms are only used to distinguish one element from another element, but do not limit the corresponding elements, irrespective of the nature or order of the corresponding elements.

Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

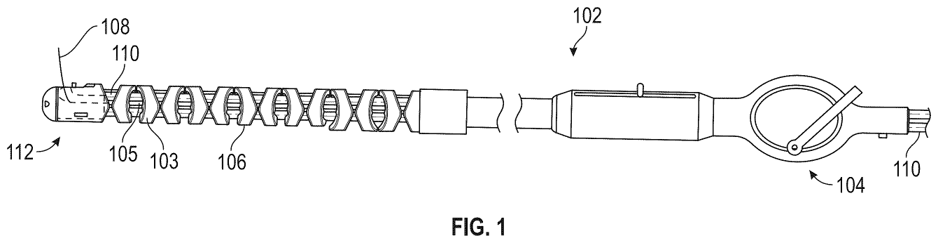

FIG. 1 shows an embodiment of a steerable medical device. The steerable medical device 102 includes a control handle 104 with a steerable outer sheath 106 extending distally therefrom. Without limitation, certain aspects of the steerable medical device 102 may include features discussed in U.S. patent application Ser. No. 16/749,083 (and corresponding Pub. No. 2020/0163534A1), which is hereby incorporated by reference in its entirety. For example, the steerable medical device 102 may generally include an endoscope (of any suitable type), and may include one or more accessory channel shafts 110. The accessory channel shafts may provide lumens or other pathways to a distal end 112 of the device 102 such that accessory components may extend to the device's distal end for performance of desired functions. For example, and without limitation, the accessory channel shafts 110 may receive a steerable device (e.g., a duodenal scope and/or a cholangioscope 108—which is a relatively small-diameter device that is best delivered to a target site via a larger catheter e.g., the outer sheath 106 and/or an accessory channel shaft).

The control handle 104 of the device 102 may be located at a proximal end of the steerable medical device 102, and therefore accessible directly by a medical professional. The handle may have any suitable features for steering the device, hereafter referred to as a steering control system 120. Certain aspects of the control handle 104 and the steering control system (e.g., control wheels) may include features described in U.S. patent application Ser. No. 15/655,239 (published as U.S. 2018/028778A1), entitled “STEERABLE MULTILUMEN CATHETER SHAFT,” which is hereby incorporated by reference in its entirety. Other features are described in U.S. patent application Ser. No. 18/589,015 (published as U.S. 2024/0293012 A1), which is hereby incorporated by reference in its entirety.

In some embodiments, small diameter instruments for use through a small-diameter working channel of the cholangioscope 108 may be used to perform a particular procedure, and such instruments have been used with success, such as biopsy forceps, cytology brushes, stone-removal devices, electrocautery devices, injection needles, and others. However, when such instruments are used with a cholangioscope, they may be limited in size (due to limited working channel size). Thus, the inventors recognize that there would be a benefit to perform the procedures with larger devices that are designed with more-optimal parameters (e.g., taking a larger biopsy sample, disintegrate a harder stone faster, removal of a larger stone, etc.). However, when using existing devices (e.g., where the second instrument is side-by-side with a cholangioscope), the control of the location and targeting of these endoscopic instruments is only possible in the forward and reverse direction, as they are not within the cholangioscope's steerable working channel, and as they typically are not steerable on their own.

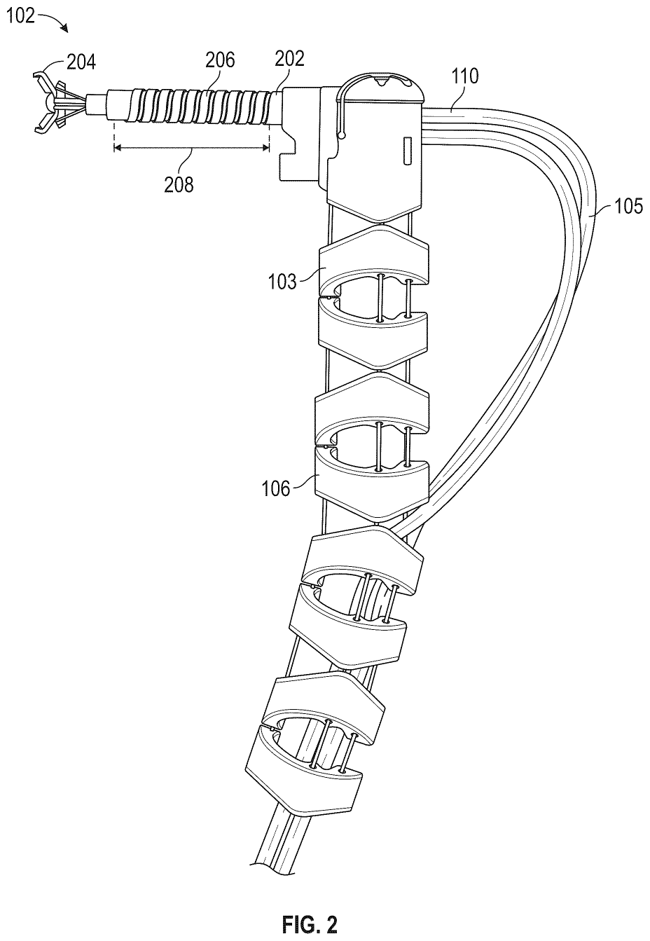

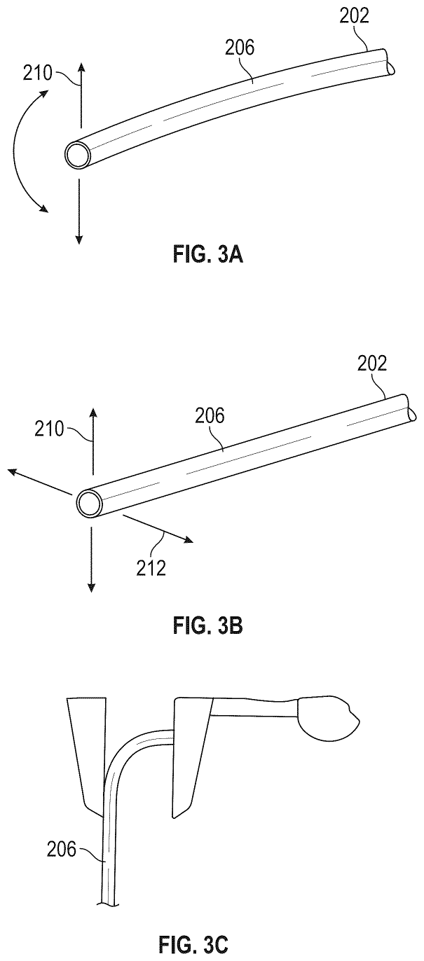

The present disclosure provides an improved device by incorporating steering into an endoscopic instrument that extends through one or more of the accessory channel shafts 110 or another working channel, particularly via the inclusion of a steerable shaft 202 as illustrated in FIGS. 2 through 3C. The steerable shaft 202 may receive an endoscopic instrument through its inner lumen, which may have a diameter (i.e., inner diameter of the steerable shaft 202) of at least about 2 mm (such as about 2.8 mm in certain advantageous embodiments). An outer diameter of the steerable shaft 202 may be less than about 5 mm (e.g., about 4 mm in certain advantageous embodiments), which allows it to move/slide proximally and distally through the respective working channel of the endoscope.

FIG. 2 shows an embodiment having a steerable medical device 102, which may be a deflectable endoscope, having the accessory channel shafts 110 in a deflected position (e.g., side-viewing configuration) at the distal end of the medical device 102. As shown, the steerable shaft 202 is in an extended state, which is a state where the distal end of the steerable shaft 202 extends out of a distal-end-opening of the respective accessory channel shaft 110 such that it is exposed. The steerable shaft 202 receives a separate medical device 204 (e.g., endoscopic instrument; surgical accessory), for example, a set of biopsy forceps, which may be operable by the operating medical professional(s) by manipulation of a handle 220 of the separate medical device 204.

As discussed in more detail below, in addition to potential for proximal and distal movement, a distal tip 206 of the steerable shaft 202 may have the ability to deflect in at least one direction. Deflection of the steerable shaft 202 may, as a result, cause control of the position of the separate medical device 204 (e.g., biopsy forceps) relative to the working channel (e.g., accessory channel shaft 110) of the endoscope with one or more degrees of freedom in addition to proximal and distal linear movement. Advantageously, the separate medical device 204 is controllable not only via the steerability of the accessory channel shafts themselves (which may be advantageous for initial deployment), but also with increased precision at a target patient site within the body. As a further enhancement, the distal tip 206 of the steerable shaft 202 may correspond with an exposed length 208 when the steerable shaft 202 is in the depicted extended position of FIG. 2 (which may be variable and controllable). For example, by increasing the exposed length 208, the potential for deflection relative to the distal terminus of the accessory channel shaft 110 may be increased, and vice versa.

FIGS. 3A and 3B show a variety of potential types of deflection of the distal tip 206 of the steerable shaft 202. As shown in FIG. 3A, the steerable shaft 202 may be steerable in a first direction 210 that is (or at least approaches) perpendicular to the longitudinal direction of the steerable shaft 202, plus is rotatable about its longitudinal axis (and herein, such rotation is included in the meaning of “deflection”). As such, it will be appreciated that by using a combination of horizontal and/or vertical deflection, a user may steer in any direction. In FIG. 3B, the steerable shaft 202 is deflectable in the first direction 210 and also a second direction 212, where the second direction 212 may be perpendicular to the longitudinal axis/direction of the steerable shaft 202 and also may be perpendicular to the first direction 210. These features from FIGS. 3A and 3B may be combined in a single embodiment, and it is contemplated that other types of deflection may also (or alternatively) be included. For illustrative purposes, FIG. 3C shows the steerable shaft 202 in a deflected state (e.g., in the first direction 210) without showing a separate endoscopic instrument.

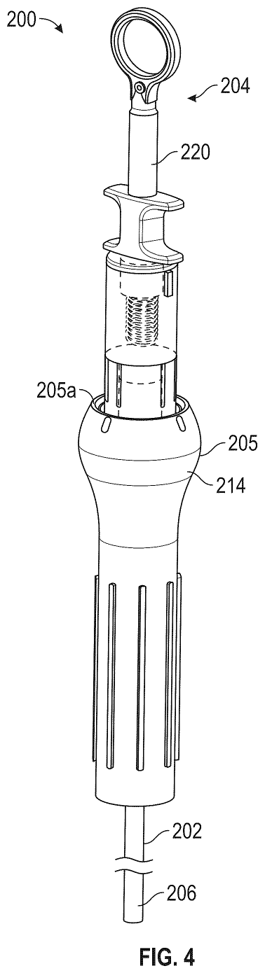

Deflection of the distal tip 206 may be achieved with any suitable device or method. For example, tip deflection may be achieved by way of deflection wires running from the distal tip 206 to the proximal end of the medical device, at an accessible position. For example, in certain embodiments and as shown by FIG. 4, the medical device may include a control assembly 200 that is dedicated to, and secured to a proximal end of, the steerable shaft 202. In other words, the control assembly 200 may be distinct from the control handle 104 shown in FIG. 1, which is particularly advantageous where the steerable shaft 202 is insertable into, and removeable from, the accessory channel shaft's proximal end, meaning that it is selectively usable depending on the need of the particular procedure.

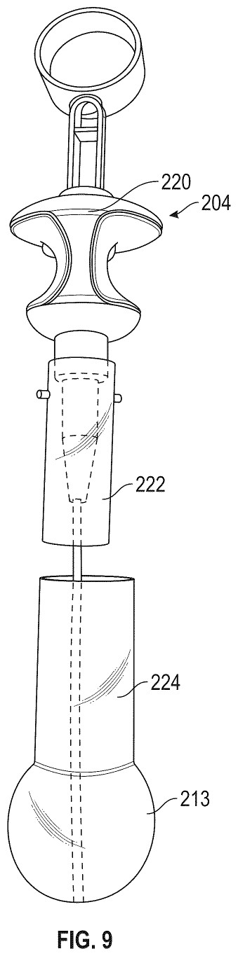

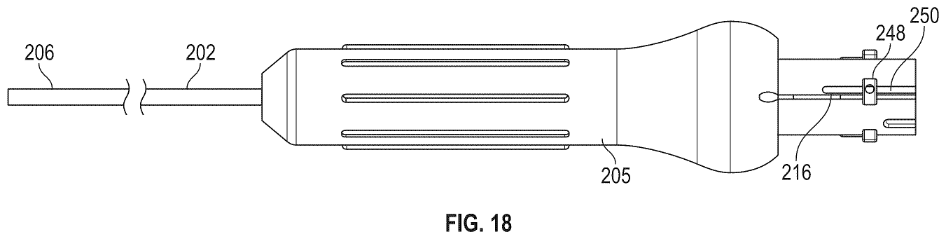

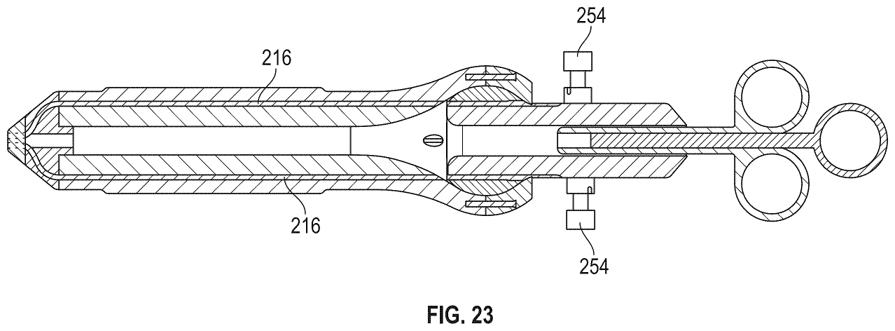

Referring to FIGS. 4-23, some embodiments of a control assembly 200 for a steerable shaft 202 (e.g., steerable medical device shaft 202) are provided. As shown in FIGS. 4 through 6, in some embodiments, the control assembly 200 includes a steerable-shaft handle 205 and a docking translating base 207 movably coupled to the steerable-shaft handle 205. The docking translating base 207 may have an elongate configuration and includes a first-end opening 209 and a second-end ball 213. As shown, the first end is a proximal end and the second end is a distal end. In other embodiments, the first end may be a distal end and the second end may be a proximal end. In some embodiments, the steerable-shaft handle 205 may include at least one port configured for irrigation, suction, injection, and/or contrast injection.

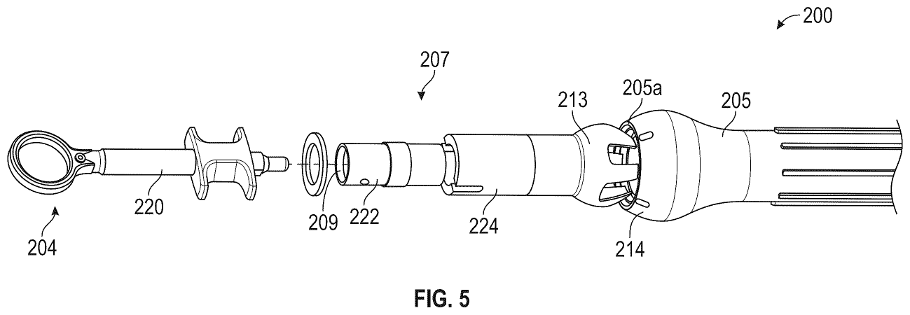

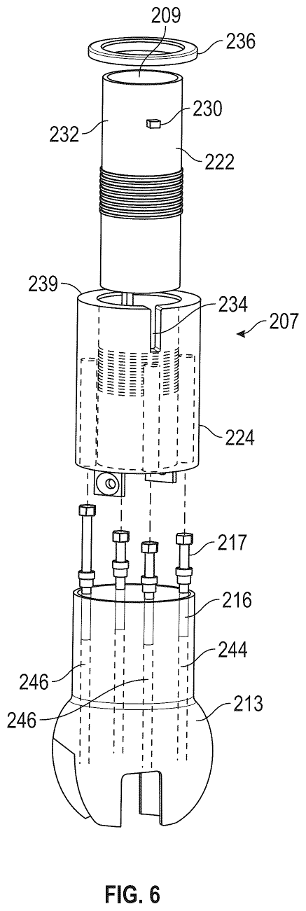

As discussed in greater detail below, the first-end opening 209 may be a lengthwise opening dimensioned to receive and operably engage (e.g., frictionally engage, press-fitting) at least a portion of a separate medical device handle 220. The separate medical device 204 may be steerable (e.g., sphincterotome, cholangioscope, by way of non-limiting example) or non-steerable (e.g., biopsy forceps, fine-needle-biopsy needle, by way of non-limiting example). In some embodiments, as shown in FIGS. 5 and 6, the second-end ball 213 has an outer diameter larger than an adjacent lengthwise portion of the base 207. Stated differently, the ball and socket arrangement shown in the drawing figures is not limiting, as those of skill in the art will understand from the present disclosure that the relative positions of the socket and the ball structures could be reversed while providing the same advantages described with reference to the illustrated embodiments herein.

Referring to FIGS. 4-6, in some embodiments, the steerable-shaft handle 205 surrounds (e.g., coaxially) a longitudinal passage extending distally from a first-end terminus 205a that includes a socket 214 dimensioned to engagingly receive the second-end ball 213. In some embodiments, the friction between the ball 213 and the socket 214 is optimized to provide a braking mechanism (e.g., to prevent inadvertent movement between the ball 213 and the socket 214) and to allow precise deflection of the distal end portion of the steerable shaft 202. In some embodiments, a brake mechanism (e.g., manual brake) may be provided to prevent inadvertent movement between the ball 213 and the socket 214.

As discussed in greater detail below, the steerable shaft 202 may be steered by the user (e.g., physician or physician's assistant) with two hands. For example, the assistant may hold the steerable-shaft handle 205 with one hand and use the other hand to articulate the ball-and-socket joint to tension the steering wires. As the steering wires'tension is altered, the distal end portion of the steerable shaft 202 will deflect. The ball-and-socket mechanism allows the assistant to deflect the distal end portion of the steerable shaft 202 360 degrees without rotating the medical device 102, and the ball-socket joint provides 360 degrees of steering in a slim and intuitive control assembly profile.

Referring to FIGS. 4-6 and 9, in some embodiments, the longitudinal passage includes a path in mechanical communication with the first-end lengthwise opening 209 of the base 207, such that a portion of the separate medical device 204 may extend through the longitudinal passage via the path when the separate medical device handle 220 is connected to the docking translating base 207 via the opening 209, as discussed above. As discussed in greater detail below, this configuration is advantageous as it allows the user (e.g., physician or physician's assistant) to manipulate the steering of the steerable shaft 202 and the use (e.g., the location and/or extension) of the separate medical device 204 (e.g., a surgical accessory, which extends through the steerable shaft 202) simultaneously via the separate medical device handle 220. In some embodiments, the base 207 may be configured to extend the distal end of the separate medical device 204 (e.g., surgical accessory) out of the distal end of the steerable shaft 202 up to a predetermined maximum distance.

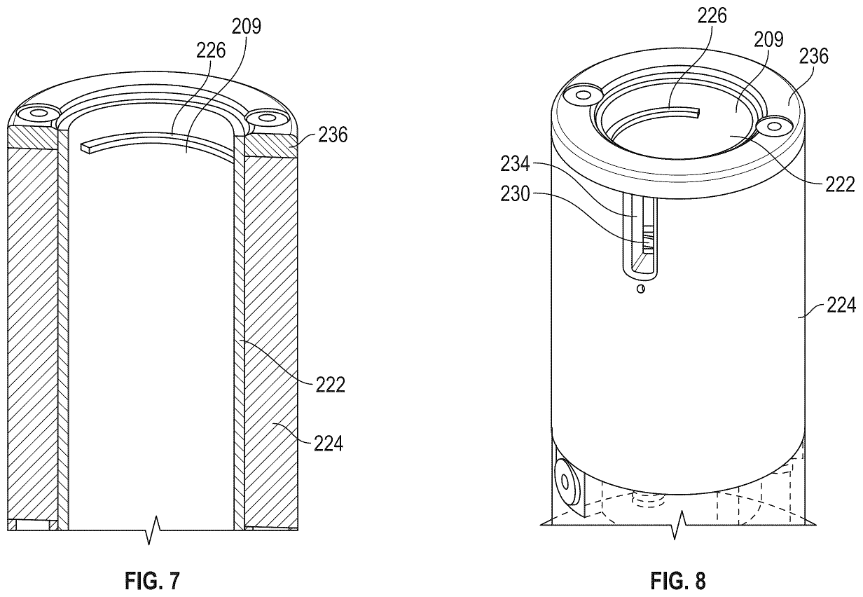

Referring to FIGS. 5-15, in some embodiments, the base 207 includes a first portion 222 and a second portion 224, where the first portion 222 is selectively moveable relative to the second portion 224 and is selectively lockable to the second portion 224. In some embodiments, as shown in FIGS. 7 and 8, the first portion 222 is at least partially disposed within the second portion 224, while in some embodiments, as shown in FIG. 11, the second portion 224 is at least partially disposed within the first portion 222. In some embodiments, as shown, the first and second portions 222 and 224 may both have a generally tubular configuration with different cross-sectional diameters. The first and second portions 222 and 224 of the base 207 may have other configurations without departing from the scope of the present invention.

In some embodiments, as shown in FIGS. 7-9, for example, the first portion 222 is configured to receive and operably engage a portion of a separate medical device handle 220, and the first portion 222 includes at least one rib 226 configured to latch onto a portion (e.g., groove) of a separate medical device handle 220. The first portion 222 and the separate medical device handle 220 may have other corresponding structures configured for forming a mechanical connection therebetween, without departing from the scope of the present invention.

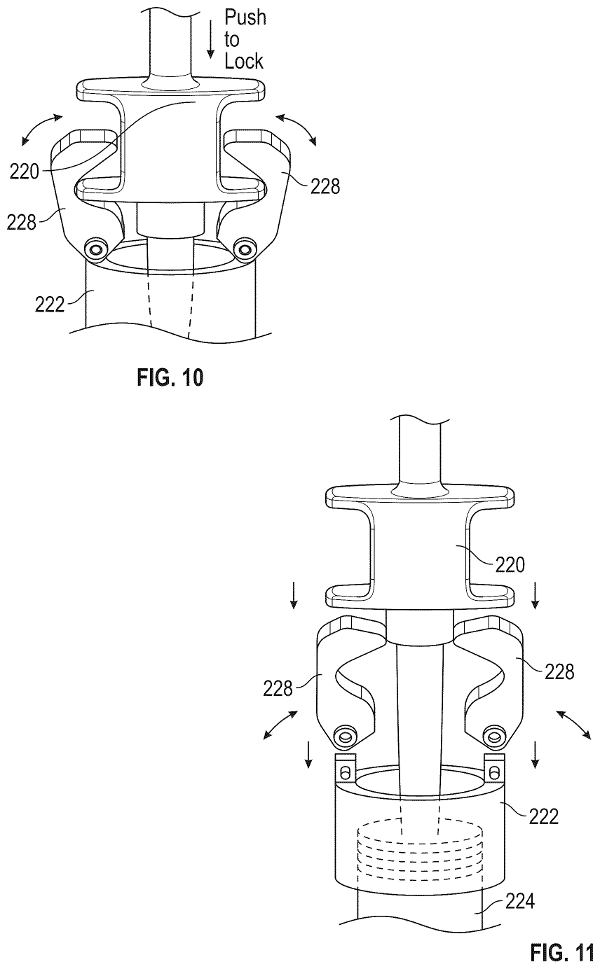

In some embodiments, as shown in FIGS. 10 and 11, at least one retractable grip 228 (e.g., two retractable grips, as shown) is connected to the first portion 222, where the at least one retractable grip 228 is configured to receive and secure at least a portion of a separate medical device handle 220 as the handle 220 is pushed into the base 207. Other suitable structures may be coupled to the first portion 222 and/or the second portion 224 of the base 207 for securing a separate medical device handle 220, without departing from the scope of the present invention (by way of non-limiting example, one or more clamps, straps, bayonet or other inter-engagement structures, or other configurations/devices may be included with or instead of the grip 228). A retrofit adapter may be provided to make the docking of the separate medical device handle 220 to the ball-and-socket joint more universal for other desired medical devices (e.g., surgical accessories).

After the separate medical device 204 (e.g., surgical accessory) is secured to the base 207, the user (e.g., physician or physician's assistant) may extend or retract the separate medical device 204 within the steerable shaft 202 to a desired distance and lock it in place to prevent inadvertent translation of the separate medical device 204.

Referring to FIGS. 6-8 and 12-15, in some embodiments, the translation of the first portion 222 relative to the second portion 224 is regulated through a tooth and groove interaction between the first portion 222 and the second portion 224. As shown, in some embodiments, the first portion 222 includes at least one tooth 230 extending radially outwardly from an outer surface 232 of the first portion 222. The second portion 224 includes at least one groove 234 extending along at least a portion of a longitudinal length of a wall 240 of the second portion 224. The at least one tooth 230 and the at least one groove 234 are configured such that the at least one tooth 230 is movably (e.g., sliding movement) received in the at least one groove 234, such that the user (e.g., physician, physician's assistant) may selectively move the first portion 222 relative to the second portion 224 via moving the at least one tooth 230 along at least a portion of the at least one groove 234, thereby moving the separate medical device 204 (which is coupled to the base 207 via the separate medical device handle 220) proximally and/or distally.

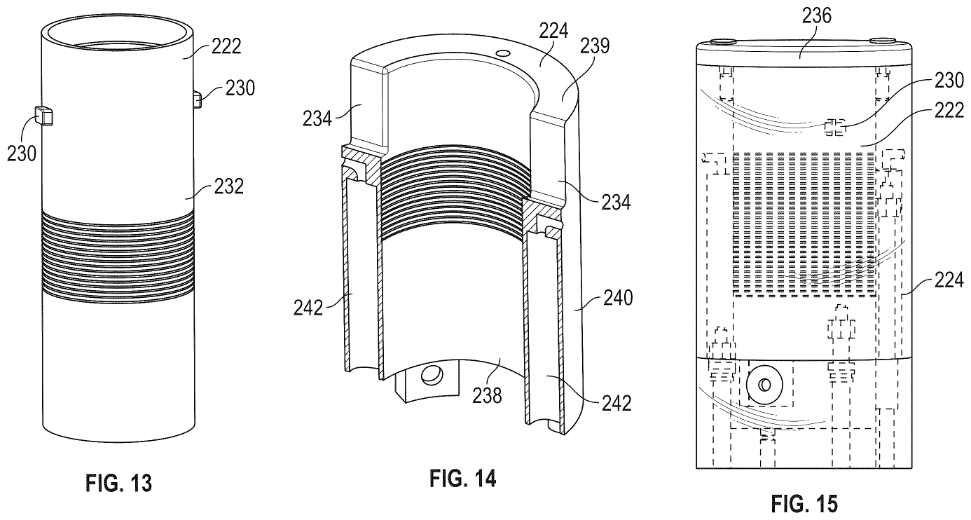

The configuration (e.g., shape, size), number, location of the at least one tooth 230 and the at least one groove 234 may be varied, as desired and/or needed, without departing the scope of the present invention. As shown in FIGS. 13 and 14, for example, the first portion 222 includes two teeth 230 disposed about 180 degrees apart (e.g., 170 degrees-190 degrees apart) on the outer surface 232 of the first portion 222. Also, the second portion 224 includes two grooves 234 disposed about 180 degrees apart (e.g., 170 degrees-190 degrees apart), configured for receiving the two teeth 230 when the first portion 222 is at least partially disposed within the second portion 224.

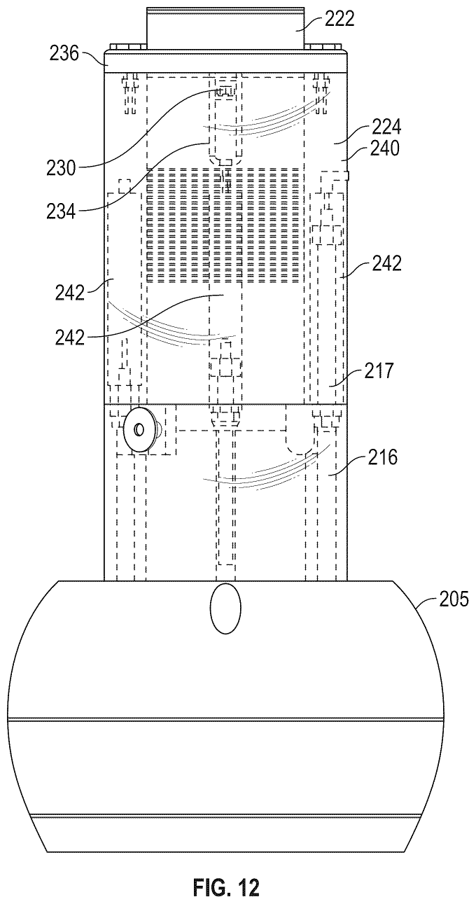

In some embodiments, a locking ring 236 may be disposed at a proximal end 239 of the second portion 224, which is configured to prevent the first portion 222 from unintentionally falling out of the second portion 224. In some embodiments, the first portion 222 is selectively locked relative to the second portion 224 via interference ribs to resist inadvertently translating (e.g., advancing or retracting) the separate medical device 204. As shown in FIGS. 12-14, for example, the outer surface 232 of the first portion 222 and the inner surface 238 of the second portion 224 may have at least one corresponding rib and groove structure. Other dampening or locking mechanisms may be used to resist inadvertent translation, without departing from the scope of the present invention.

The translation mechanism (e.g., tooth and groove interaction) between the first portion 222 and the second portion 224 along with the dampening or locking mechanism between the first portion 222 and the second portion 224 provide a controlled translation mechanism that gives the user the ability to selectively advance and retract the separate medical device 204 to desired locations.

In some embodiments, as shown in FIGS. 6, 12, 15, 17 and 18 for example, the base 207 includes at least two steering wires 216 (e.g., four steering wires as shown in FIG. 6) that extend through the steerable-shaft handle 205 in mechanical communication with a steerable length of a shaft length disposed distal of the steerable-shaft handle 205. As shown, the wall 240 of the second portion 224 includes at least two lumens 242 and the wall 244 of the ball 213 includes at least two lumens 246, which are configured to receive the proximal end portions of the at least two steering wires extending therethrough. In some embodiments, the steerable shaft 202 includes at least two lumens (e.g., four lumens) that each house a steering wire of the at least two steering wires 216, and the at least two steering wires 216 are anchored onto a distal end portion of the steerable shaft 202. The at least two steering wires 216 extend past the proximal end of the steerable shaft 202 and are anchored in the control assembly 200 (e.g., in the lumens 242 of the second portion 224, as shown in FIG. 12).

In some embodiments, the control assembly 200 may also include a tensioning control mechanism configured for adjusting tensioning of the steering wires to optimize individual wire tension and enhance steering performance. While these tensioning may be used during production to optimize tensioning, they may also serve beneficial in a procedure. For example, as a physician navigates the tortious anatomy of the biliary tree, the deflection of each steering wire may become inadequate due to differential hysteresis of each of the steering wires. The tensioning control mechanism allows the user to adjust the necessary tensioning for adequate steering.

In some embodiments, as shown in FIGS. 6 and 12, the tensioning control mechanism includes anchoring each steering wire 216 to a screw 217, and each screw 217 is threaded into a lumen 242 in the wall 240 of the second portion 224. The extension or retraction of the screw 217 from or into the lumen 242 adjusts the tension of the steering wire 216.

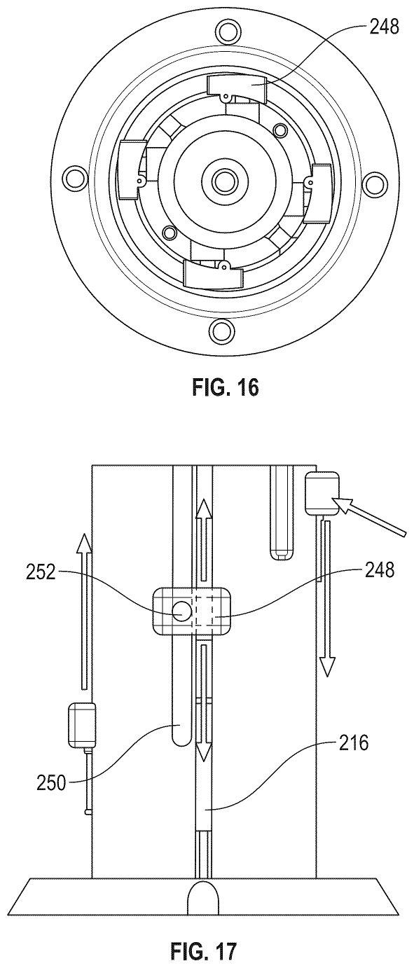

In some embodiments, as shown in FIGS. 16-18, the tensioning control mechanism includes a plurality of (e.g., four) sliders 248 and each slider 248 is attached to a proximal end portion of a steering wire 216. A plurality of sliding tracks (grooves) may be provided on an outer surface of the base 207 (e.g., the outer surface of the second portion 224) such that at least a portion of each slider 248 may be moved along a respective sliding track 250 (e.g., proximally or distally) to increase or decrease wire tension.

Each slider 248 can be locked in place at a selected location along a respective sliding track 250, via any suitable means (e.g., via a screw hole 252), thereby preventing inadvertent sliding.

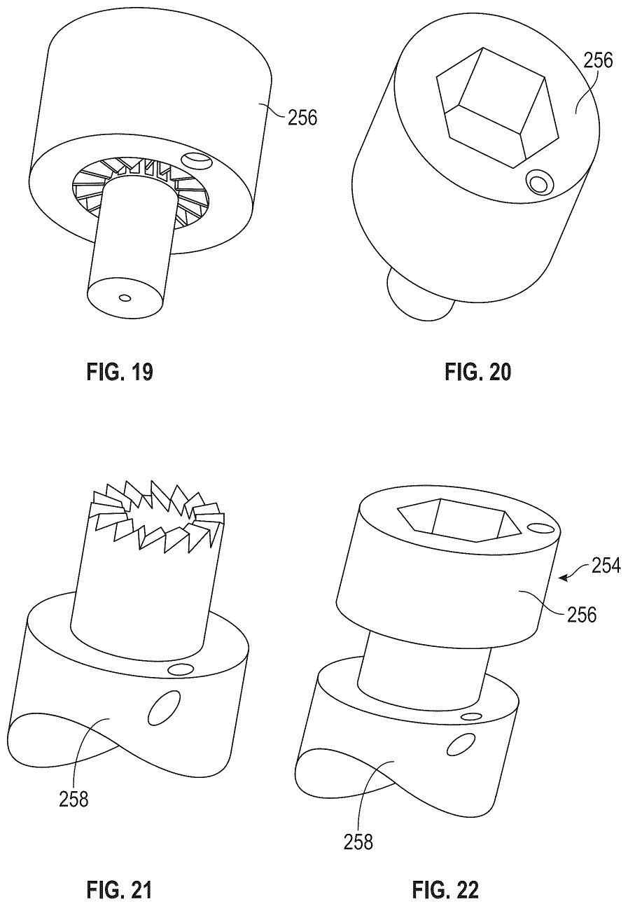

In some embodiments, as shown in FIGS. 19-23, the tensioning control mechanism includes a plurality of anchors 254 and each anchor 254 is attached to a proximal end portion of a steering wire 216. As shown, the anchor 254 includes an upper portion 256 and a lower portion 258, where the upper portion 256 is rotatable relative to the lower portion 258. The upper portion 256 and the lower portion 258 may have corresponding tooth and groove structures, such that the upper portion 256 may be rotated relative to the lower portion 258 and be locked to the lower portion at a desired location via a tooth and groove interaction. In some embodiments, the lower portion 258 may be secured at a desired location on the base 207, and the proximal end portion of the steering wire 216 is attached to the upper portion 256, such that a user may manually turn the upper portion 256 to wrap and tension the steering wire 216.

Referring to FIGS. 1-4, when the steerable shaft 202 and the control assembly 200 are used with the medical device 102, a scope system is formed, which includes a steerable outer sheath 106 having a lumen extending therethrough and an articulating distal lengthwise portion 103, and an accessory channel shaft 110 having an accessory lumen extending therethrough and a distal lengthwise section 105. The accessory channel shaft 110 is movably disposed at least partially within the lumen of the steerable outer sheath 106. The accessory channel shaft 110 is moveable between a forward-viewing configuration (e.g., as shown in FIG. 1) and a side-viewing configuration (e.g., as shown in FIG. 2) relative to the steerable outer sheath 106.

In the forward-viewing configuration, the distal lengthwise section 105 of the accessory channel shaft 110 is parallel to and/or disposed at least partially within the distal lengthwise portion 103 of the steerable outer sheath 106, and in the side-viewing configuration, the distal lengthwise section 105 of the accessory channel shaft 110 is disposed at a non-parallel angle relative to the distal lengthwise portion 103 of the steerable outer sheath 106.

A steerable medical device shaft 202 is movably disposed in the accessory channel shaft 110 (e.g., when the accessory channel shaft 110 is in the forward-viewing configuration and/or in the side-viewing configuration), where the steerable medical device shaft 202 is controlled by the control assembly 200, as discussed above. In some embodiments, visual/tactile marks may be provided on the steerable medical device shaft 202 for scope alignment, including but not limited to denoting when the distal end of the steerable medical device shaft 202 exits the scope channel (e.g., the accessory channel shaft 110). The control assembly 200 includes a steerable-shaft handle 205, which is connected to a docking translating base 207, and the base 207 includes a first-end opening 209 dimensioned to receive and operably engage at least a portion of a separate medical device handle 220.

The present invention is advantageous as it can be controlled by a physician's assistant, allowing the physician to focus on endoscope operations. Further, the control assembly has a small profile, and is intuitive to control via a ball-socket joint, and is easy to handle due to the capability to dock the separate medical device handle (e.g., handle of forceps or other surgical accessories) directly to the control assembly.

While various embodiments have been described, it will be apparent to those of ordinary skill in the art that many more embodiments and implementations are possible. Accordingly, the embodiments described herein are examples, not the only possible embodiments and implementations.

Having described various aspects of the subject matter above, additional disclosure is provided below that may be consistent with the claims originally filed with this disclosure. In describing this additional subject matter, reference may be made to the previously described figures. Any of the following aspects may be combined, where compatible.

A first aspect relates to a control assembly for a steerable medical device shaft, the control assembly comprising: a steerable-shaft handle; and an elongate docking translating base, including a first-end lengthwise opening dimensioned to receive and operably engage a portion of a separate medical device handle and a second-end ball with an outer diameter larger than an adjacent lengthwise portion of the base, wherein the steerable-shaft handle coaxially surrounds a longitudinal passage extending distally from a first-end terminus that includes a socket dimensioned to engagingly receive the second-end ball, the longitudinal passage including a path in mechanical communication with the first-end lengthwise opening of the base, and wherein the base includes at least two steering wires that extend through the steerable-shaft handle in mechanical communication with a steerable length of a shaft length disposed distal of the steerable-shaft handle.

A second aspect relates to the control assembly of aspect 1, wherein the separate medical device is non-steerable.

A third aspect relates to the control assembly of any preceding aspect, wherein the first end is a proximal end and the second end is a distal end.

A fourth aspect relates to the control assembly of any preceding aspect, wherein the steerable shaft includes at least two lumens that each house a steering wire of the at least two steering wires, and wherein the at least two steering wires are anchored onto a distal end of the steerable shaft.

A fifth aspect relates to the control assembly of any preceding aspect, wherein the base includes a first portion and a second portion, the first portion being selectively moveable relative to the second portion and selectively lockable to the second portion.

A sixth aspect relates to the control assembly of any preceding aspect, wherein the first portion is configured to receive and operably engage a portion of a separate medical device handle, and wherein the first portion includes at least one rib configured to latch onto a portion of a separate medical device handle.

A seventh aspect relates to the control assembly of any preceding aspect, further comprising at least one retractable grip connected to the first portion, wherein the at least one retractable grip is configured to receive and secure a portion of a separate medical device handle.

An eighth aspect relates to the control assembly of any preceding aspect, wherein translation of the first portion relative to the second portion is regulated through a tooth and groove interaction between the first portion and the second portion.

A ninth aspect relates to the control assembly of any preceding aspect, wherein the first portion is locked relative to the second portion via interference ribs.

A tenth aspect relates to the control assembly of any preceding aspect, wherein the first portion is at least partially disposed within the second portion.

An eleventh aspect relates to the control assembly of any preceding aspect, further comprising a tensioning control mechanism configured for adjusting tensioning of the at least two steering wires.

A twelfth aspect relates to a control assembly for a steerable medical device shaft, the control assembly comprising: a steerable-shaft handle; and an elongate docking translating base, including a first-end lengthwise opening dimensioned to receive and frictionally engage a portion of a separate medical device handle and a second-end ball with an outer diameter larger than an adjacent lengthwise portion of the base, wherein the elongate docking translating base includes a first portion and a second portion, the first portion being selectively moveable relative to the second portion and selectively lockable to the second portion, wherein the steerable-shaft handle surrounds a longitudinal passage extending distally from a first-end terminus that includes a socket dimensioned to engagingly receive the second-end ball, and wherein the base includes at least two steering wires that extend through the steerable-shaft handle in mechanical communication with a steerable length of a shaft length disposed distal of the steerable-shaft handle.

A thirteenth aspect relates to the control assembly of aspect 12, wherein the steerable shaft includes at least two lumens that each house a steering wire of the at least two steering wires, and wherein the at least two steering wires are anchored onto a distal end of the steerable shaft.

A fourteenth aspect relates to the control assembly of any one of aspects 12 or 13, wherein the first portion is configured to receive and operably engage a portion of a separate medical device handle, and wherein the first portion includes at least one rib configured to latch onto a portion of a separate medical device handle.

A fifteenth aspect relates to the control assembly of any one of aspects 12 to 14, further comprising at least one retractable grip connected to the first portion, wherein the at least one retractable grip is configured to receive and secure a portion of a separate medical device handle.

A sixteenth aspect relates to the control assembly of any one of aspects 12 to 15, wherein the first portion is at least partially disposed within the second portion.

A seventeenth aspect relates to a scope system, comprising: a steerable outer sheath comprising a lumen extending therethrough and an articulating distal lengthwise portion; and an accessory channel shaft comprising an accessory lumen extending therethrough and a distal lengthwise section, the accessory channel shaft movably disposed at least partially within the lumen of the steerable outer sheath, wherein the accessory channel shaft is moveable between a forward-viewing configuration and a side-viewing configuration relative to the steerable outer sheath, wherein, in the forward-viewing configuration, the distal lengthwise section of the accessory channel shaft is parallel to and/or disposed at least partially within the distal lengthwise portion of the steerable outer sheath, wherein, in the side-viewing configuration, the distal lengthwise section of the accessory channel shaft is disposed at a non-parallel angle relative to the distal lengthwise portion of the steerable outer sheath, wherein a steerable medical device shaft is movably disposed in the accessory channel shaft, and wherein the steerable medical device shaft is controlled by a control assembly including a steerable-shaft handle, the steerable-shaft handle is connected to a docking translating base, and the base includes a first-end opening dimensioned to receive and operably engage a portion of a separate medical device handle.

An eighteenth aspect relates to the scope system of aspect 17, wherein the base includes a second-end ball, wherein the steerable-shaft handle coaxially surrounds a longitudinal passage extending distally from a first-end terminus that includes a socket dimensioned to engagingly receive the second-end ball, and wherein the base includes at least two steering wires that extend through the steerable-shaft handle in mechanical communication with a steerable length of a shaft length disposed distal of the steerable-shaft handle.

A nineteenth aspect relates to the scope system of any one of aspects 17 or 18, wherein the base includes a first portion and a second portion, the first portion being selectively moveable relative to the second portion and selectively lockable to the second portion.

A twentieth aspect relates to the scope system of any one of aspects 17 to 19, wherein the steerable shaft includes at least two lumens that each house a steering wire of the at least two steering wires, and wherein the at least two steering wires are anchored onto a distal end of the steerable shaft.

Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they are used in a generic and descriptive sense only and not for purposes of limitation unless specifically defined by context, usage, or other explicit designation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment. In the event of any inconsistent disclosure or definition from the present application conflicting with any document incorporated by reference, the disclosure or definition herein shall be deemed to prevail.

We claim:

1. A control assembly for a steerable medical device shaft, the control assembly comprising:
a steerable-shaft handle; and
an elongate docking translating base, including a first-end lengthwise opening dimensioned to receive and operably engage a portion of a separate medical device handle and a second-end ball with an outer diameter larger than an adjacent lengthwise portion of the base,
wherein the steerable-shaft handle coaxially surrounds a longitudinal passage extending distally from a first-end terminus that includes a socket dimensioned to engagingly receive the second-end ball, the longitudinal passage including a path in mechanical communication with the first-end lengthwise opening of the base, and
wherein the base includes at least two steering wires that extend through the steerable-shaft handle in mechanical communication with a steerable length of a shaft length disposed distal of the steerable-shaft handle.

2. The control assembly of claim 1, wherein the separate medical device is non-steerable.

3. The control assembly of claim 1, wherein the first end is a proximal end and the second end is a distal end.

4. The control assembly of claim 1, wherein the steerable shaft includes at least two lumens that each house a steering wire of the at least two steering wires, and wherein the at least two steering wires are anchored onto a distal end of the steerable shaft.

5. The control assembly of claim 1, wherein the base includes a first portion and a second portion, the first portion being selectively moveable relative to the second portion and selectively lockable to the second portion.

6. The control assembly of claim 5, wherein the first portion is configured to receive and operably engage a portion of a separate medical device handle, and wherein the first portion includes at least one rib configured to latch onto a portion of a separate medical device handle.

7. The control assembly of claim 5, further comprising at least one retractable grip connected to the first portion, wherein the at least one retractable grip is configured to receive and secure a portion of a separate medical device handle.

8. The control assembly of claim 5, wherein translation of the first portion relative to the second portion is regulated through a tooth and groove interaction between the first portion and the second portion.

9. The control assembly of claim 5, wherein the first portion is locked relative to the second portion via interference ribs.

10. The control assembly of claim 5, wherein the first portion is at least partially disposed within the second portion.

11. The control assembly of claim 1, further comprising a tensioning control mechanism configured for adjusting tensioning of the at least two steering wires.

12. A control assembly for a steerable medical device shaft, the control assembly comprising:
a steerable-shaft handle; and
an elongate docking translating base, including a first-end lengthwise opening dimensioned to receive and frictionally engage a portion of a separate medical device handle and a second-end ball with an outer diameter larger than an adjacent lengthwise portion of the base,
wherein the elongate docking translating base includes a first portion and a second portion, the first portion being selectively moveable relative to the second portion and selectively lockable to the second portion,
wherein the steerable-shaft handle surrounds a longitudinal passage extending distally from a first-end terminus that includes a socket dimensioned to engagingly receive the second-end ball, and
wherein the base includes at least two steering wires that extend through the steerable-shaft handle in mechanical communication with a steerable length of a shaft length disposed distal of the steerable-shaft handle.

13. The control assembly of claim 12, wherein the steerable shaft includes at least two lumens that each house a steering wire of the at least two steering wires, and wherein the at least two steering wires are anchored onto a distal end of the steerable shaft.

14. The control assembly of claim 12, wherein the first portion is configured to receive and operably engage a portion of a separate medical device handle, and wherein the first portion includes at least one rib configured to latch onto a portion of a separate medical device handle.

15. The control assembly of claim 12, further comprising at least one retractable grip connected to the first portion, wherein the at least one retractable grip is configured to receive and secure a portion of a separate medical device handle.

16. The control assembly of claim 12, wherein the first portion is at least partially disposed within the second portion.

17. A scope system, comprising:
a steerable outer sheath comprising a lumen extending therethrough and an articulating distal lengthwise portion; and
an accessory channel shaft comprising an accessory lumen extending therethrough and a distal lengthwise section, the accessory channel shaft movably disposed at least partially within the lumen of the steerable outer sheath, wherein the accessory channel shaft is moveable between a forward-viewing configuration and a side-viewing configuration relative to the steerable outer sheath,
wherein, in the forward-viewing configuration, the distal lengthwise section of the accessory channel shaft is parallel to and/or disposed at least partially within the distal lengthwise portion of the steerable outer sheath, wherein, in the side-viewing configuration, the distal lengthwise section of the accessory channel shaft is disposed at a non-parallel angle relative to the distal lengthwise portion of the steerable outer sheath,
wherein a steerable medical device shaft is movably disposed in the accessory channel shaft, and
wherein the steerable medical device shaft is controlled by a control assembly including a steerable-shaft handle, the steerable-shaft handle is connected to a docking translating base, and the base includes a first-end opening dimensioned to receive and operably engage a portion of a separate medical device handle.

18. The scope system of claim 17, wherein the base includes a second-end ball,
wherein the steerable-shaft handle coaxially surrounds a longitudinal passage extending distally from a first-end terminus that includes a socket dimensioned to engagingly receive the second-end ball, and
wherein the base includes at least two steering wires that extend through the steerable-shaft handle in mechanical communication with a steerable length of a shaft length disposed distal of the steerable-shaft handle.

19. The scope system of claim 17, wherein the base includes a first portion and a second portion, the first portion being selectively moveable relative to the second portion and selectively lockable to the second portion.

20. The scope system of claim 17, wherein the steerable shaft includes at least two lumens that each house a steering wire of the at least two steering wires, and wherein the at least two steering wires are anchored onto a distal end of the steerable shaft.
